(19) United States
(12) Patent Application Publication
(10) Pub. No.: US 2022/0049317 A1
(43) Pub. Date: Feb. 17, 2022

(54) IN-VITRO METHOD FOR DIAGNOSIS AND PROGNOSIS OF A DISEASE

(71) Applicant: HELMHOLTZ ZENTRUM MÜNCHEN - DEUTSCHES FORSCHUNGSZENTRUM FÜR GESUNDHEIT UND UMWELT (GMBH), Neuherberg (DE)

(72) Inventors: Axel WALCH, Baldham (DE); Katharina HUBER, Tacherting (DE); Achim BUCK, München (DE); Thomas KUNZKE, München (DE)

(21) Appl. No.: 17/413,275

(22) PCT Filed: Dec. 10, 2019
(86) PCT No.: PCT/EP2019/084475

§ 371 Date: Jun. 11, 2021

(30) Foreign Application Priority Data
Dec. 11, 2018 (LU) LU101032

(51) Int. Cl. C12Q 1/6886; C12Q 1/6841; G01N 1/30

(52) U.S. Cl. CPC C12Q 1/6886; C12Q 1/6841; C12Q 2600/118; C12Q 2600/156; G01N 1/30

(57) ABSTRACT

The present invention relates an in-vitro method for diagnosis and/or prognosis of a disease in a tissue sample obtained from a mammalian subject comprising (i) Determining the level of a first biomarker using matrix-assisted laser desorption/ionization (MALDI)-imaging in the sample; (ii) Performing a histochemical staining of the sample; (iii) Determining the ratio of the levels of a second biomarker and a reference marker using fluorescence in situ-hybridization (FISH); wherein the same sample is used in steps (i) to (iii).

### TECHNICAL FIELD OF THE INVENTION

The present invention relates to an in-vitro method for diagnosis and/or prognosis of diseases in a tissue sample obtained from a mammalian subject comprising performing MALDI imaging, a histochemical staining of the sample and a FISH analysis of the very same sample.

### BACKGROUND

The diagnosis and prognosis of disease often relies on the analysis of tissue samples obtained from a subject suspected of suffering from a disease. Such tissue samples may be biopsies that have been obtained from a patient, and are in general fixed on a glass slide for further analysis. In general, such samples are first analyzed by a pathologist using a light microscope. Beside the simple view of the tissues under a light microscope without any further staining, many different staining methods for highlighting the specific morphology of the tissue samples have been developed, along with more advanced microscope technology.

Histochemical staining is the oldest technique used in microscopy to enhance contrast in the microscopic image. For example, the silver stain of Camillo Golgi was published in 1873. Stains and dyes are frequently used to highlight structures in biological tissues for viewing. Stains may be used to define and examine bulk tissues (highlighting, for example, muscle fibers or connective tissue), cell populations (classifying different blood cells, for instance), or organelles within individual cells. Simple staining is staining with only one stain/dye. There are various kinds of multiple staining, many of which are examples of counterstaining, differential staining, or both, including double staining and triple staining.

Histochemical stainings include the Gram staining to differentiate between gram-positive and gram-negative bacteria, the hematoxylin and eosin (H&E) staining that helps to differentiate between nuclei and cytoplasm or the Papanicolaou staining that is frequently used in the diagnosis of cancer. Although many of these technologies have been used for several decades, the analysis of stained tissue section remains very subjective. The use of histochemical stainings enables the pathologist to compare the structure of cells in a tissue section from a patient with a healthy subject and to use these to diagnose the disease.

The development of immunohistochemical staining helped to highlight very specific tissue structures and allowed staining with higher resolution and specificity. Immunohistochemistry (IHC), sometimes also called immunostaining, involves the process of selectively imaging antigens in cells of a tissue section by exploiting the principle of antibodies binding specifically to antigens in biological tissues. Albert Coons conceptualized and first implemented the procedure in 1941. Immunohistochemical staining is widely used in the diagnosis of abnormal cells such as those found in cancerous tumors. Specific molecular markers are characteristic of particular cellular events such as proliferation or cell death (apoptosis). Immunohistochemistry is also widely used in basic research to understand the distribution and localization of biomarkers and differentially expressed proteins in different parts of a biological tissue. Visualizing an antibody-antigen interaction can be accomplished in a number of ways. In the most common instance, an antibody is conjugated to an enzyme, such as peroxidase, that can catalyze a color-producing reaction. Alternatively, the antibody can also be tagged to a fluorophore, such as fluorescein or rhodamine (immunofluorescence).

Fluorescence in situ hybridization (FISH) has been developed as another tool for the analysis of tissue sections by microscopy. Instead of using antibodies to target any antigen in the cells, FISH uses fluorescent probes that bind to only those parts of the chromosome with a high degree of sequence complementarity. It was developed by biomedical researchers in the early 1980s and is used to detect and localize the presence or absence of specific DNA sequences on chromosomes. Fluorescence microscopy can be used to find out where the fluorescent probe is bound to the chromosomes. FISH is often used for identifying specific features in DNA for use in genetic counseling, medicine, and species identification. FISH can also be used to detect and localize specific RNA targets (mRNA, IncRNA and miRNA) in cells, circulating tumor cells, and tissue samples. In this context, it can help define the spatial-temporal patterns of gene expression within cells and tissues.

FISH is also used in the diagnosis diseases or the stratification whether a patient will profit from a specific therapy. For example, 10-20% of breast cancer and gastric cancer cases show an overexpression of the human epidermal growth factor receptor 2 (HER2). Cancer patients who are HER2 receptor-positive can be treated with the antibody Trastuzumab (Herceptin™) which is directed against the extracellular domain of HER2. Tissue sections from patients that show an increased number of HER2 copies in cells are most likely more vulnerable to a therapy with Trastuzumab.

However, all these optical tests bear the risk of false positive and false negative test results in tissue testing. Immunohistochemistry and in situ hybridization are both difficult to manage and subject to interpretation. The reproducibility, especially in immunohistochemistry, is often poor. In situ hybridization is a method which allows quantification of gene copy numbers, the interpretation of which is merely performed by the naked human eye resulting in unwanted subjectivity. Thus, there is the need to add further information to the results obtained by microscopic interpretation of tissue sections to improve the diagnosis and/or prognosis of diseases. This information should be directly correlated or co-registered to the results of the histochemical staining and the FISH to allow an overlay of the information. The inventors therefore sought for a method of in-vitro analysis of tissue samples that would provide further information from tissue samples. As the necessary technology was available in the laboratory facilities, the inventors decided to try MALDI (matrix-assisted laser desorption/ionization) imaging on their tissue samples, not really expecting this to be successful.

“MALDI” itself (matrix-assisted laser desorption/ionization) is an ionization technique for mass spectrometry. This technique uses a laser energy absorbing matrix to create ions from large molecules with minimal fragmentation. It may be applied to the analysis of biomolecules (biopolymers such as DNA, proteins, peptides and sugars) and large organic molecules (such as polymers, dendrimers and other macromolecules. It is similar in character to electrospray ionization (ESI) in that both techniques are relatively soft (low fragmentation) ways of obtaining ions of large molecules in the gas phase, though MALDI typically produces far fewer multi-charged ions. Typically, MALDI methodology is a three-step process. First, the sample is mixed with a suitable matrix material and applied to a metal plate. Second, a pulsed laser irradiates the sample, triggering ablation and desorption of the sample and matrix material. Finally, the analyte molecules are ionized by being protonated or deprotonated in the hot plume of ablated gases, and then they can be accelerated into whichever mass spectrometer is used to analyze them. However, “classical” MALDI is not suitable for tissue samples.

As a further development of MALDI, MALDI imaging has been developed. MALDI imaging mass spectrometry (MALDI-IMS) or MALDI imaging is the use of matrix-assisted laser desorption ionization as a mass spectrometry imaging technique in which the sample, often a thin tissue section, is moved in two dimensions while the mass spectrum is recorded. MALDI imaging allows analyzing the spatial distribution of metabolites in a cell. The 2D spectrum may be then transformed into a heat map for each m/z species and correlated with an optical image of the sample that was taken before the MALDI imaging. However, MALDI imaging uses a UV laser beam to ionize the tissue section and it is well-known that UV light may affect nucleic acids.

Surprisingly, the inventors found that MALDI imaging did not destroy the tissue sample as expected but allowed a subsequent analysis by histochemical staining and FISH.

### SUMMARY OF THE INVENTION

The present invention relates to an in-vitro method for diagnosis and/or prognosis of disease in a tissue sample obtained from a mammalian subject comprising
(i) Determining the level of a first biomarker using matrix-assisted laser desorption/ionization (MALDI)-imaging in the sample;
(ii) Performing a histochemical staining of the sample including digitalization;
(iii) Determining the ratio of the levels of a second biomarker and a reference marker using fluorescence in situ-hybridization (FISH);
wherein the same tissue sample is used in steps (i) to (iii),
wherein the level of the first biomarker is analyzed in regions of the sample, which are suspicious for the disease as found by staining of step (ii), and wherein a deviation of the level of the first biomarker, and a deviation of the ratio of the second biomarker and the reference marker in comparison to a tissue sample of a healthy subject is indicative for the presence of the disease.

In one embodiment, the tissue sample is a formalin-fixed paraffin-embedded (FFPE) sample.

In one embodiment, the sample is deparaffinized prior to step (i).

In one embodiment, the sample has been coated with a MALDI matrix prior to step (i) preferably after deparaffinization, wherein the MALDI matrix comprises 9-aminoacridine hydrochloride monohydrate.

In one embodiment, the sample has been coated with chemical substances for derivatization prior to step (i) preferably after deparaffinization.

In one embodiment, the sample has been scanned to acquire tissue images for co-registration, and/or image analysis purposes prior to step (i).

In one embodiment, the MALDI matrix is removed after step (i), preferably by washing with 70% ethanol.

In one embodiment, the sample is washed after step (ii).

In one embodiment, the in-vitro method further comprises the determination of the level of at least one additional biomarker, preferably the in-vitro method further comprises the determination of the level(s) of 1, 2, 3, 4, 5, 6, 7, 8, 9, 10 or more than 10 additional biomarkers in step (i).

In one embodiment, the disease is cancer and the first and/or the second biomarker is a tumor marker.

In one embodiment, the first biomarker is adenosine monophosphate (AMP), wherein an increased AMP level is indicative for a diagnosis of cancer.

In one embodiment, the second marker is HER2/neu (locus 17q11.2-q12) and the reference marker is the a satellite DNA sequence at the centromeric region of chromosome 17 (CEP17; locus 17p11.1-q11.1) and wherein a ratio of Her2/neu to CEP17 is indicative for a diagnosis of cancer.

In one embodiment, the first biomarker is adenosine monophosphate (AMP), the staining in step (ii) is hematoxylin and eosin stain (H&E stain), the second biomarker is HER2/neu and the reference marker is CEP17 and wherein an increase of the ratio of AMP to the ratio of HER2/neu and CEP17 is indicative for the presence of a cancer or a tumor precursor lesion.

In one embodiment, the disease is breast cancer, Barrett's cancer or gastroesophageal adenocarcinoma.

In one embodiment, the disease is a degenerative or inflammatory disease.

The inventors surprisingly found that the same sample such as a tissue section may be analyzed by MALDI imaging and then still can be used for a FISH analysis (see Example 1). The combination of MALDI imaging followed by FISH analysis therefore provides an improved in vitro method of diagnosis and/or prognosis of a disease in a tissue sample. Regarding the fact that the molecules are ionized from the tissue by an UV laser beam, it is surprising that the DNA is still intact to allow reliable FISH results after the MALDI imaging.

### BRIEF DESCRIPTION OF THE DRAWINGS

The invention will be better understood with reference to the detailed description when considered in conjunction with the non-limiting examples and the accompanying drawings, in which:

FIG. 1 shows an exemplary workflow of MALDI imaging with hematoxylin and eosin (H&E) staining, followed by a FISH analysis of the very same tissue section.

FIG. 2 shows biopsies of gastroesophageal adenocarcinoma patients that were measured using matrix-assisted laser desorption/ionization Fourier-transform ion cyclotron resonance mass spectrometry (MALDI FT-ICR) imaging for the analysis of metabolites followed by H&E staining. The MALDI visualization of molecules is depicted using heat map coloring in the first block. The MALDI imaging heat map pictures are displayed as merged figures with the H&E stainings as background to enable morphologic correlation. FISH analysis of both biopsies was performed subsequent to the MALDI imaging procedure. For validation, consecutive sections of the very same biopsies were analyzed by FISH exclusively.

FIG. 3 shows HER2/neu signals, CEP17 signals, and HER2-CEP17 ratios derived from FISH analysis of 2 human gastroesophageal cancer biopsies.

FIG. 4 shows a tissue microarray (TMA) containing 69 human gastroesophageal adenocarcinoma samples measured by MALDI FT-ICR metabolome imaging. Several molecules are visualized exemplarily on 6 tissue cores in heat map according to the biopsies, which are displayed in FIG. 2.

FIG. 5 shows FISH analysis after H&E staining performed on the very same TMA section. The nuclei are stained using DAPI (grey).

FIG. 6 shows a Kaplan-Meier survival analysis that was done using the parameters HER2/CEP17 signal ratio from FISH analysis (A), adenosine monophosphate (AMP) mass signal intensity from the MALDI imaging approach (B), and ratio of both, AMP mass intensity/(HER2/CEP17) (C).

FIG. 7 shows a Spearman correlation of AMP level detected by MALDI imaging and HER2/neu signal count derived from FISH testing of a tissue microarray containing 69 human gastroesophageal adenocarcinoma tissues.

### DEFINITIONS

“In vitro” as used herein relates to a method that is performed outside of the body of a mammalian subject. For example, the tissue sample used for the in-vitro method of the present invention is outside of its natural context and does not include any manipulation of the mammalian subject itself. In vitro in the present case also refers to an “ex vivo” method. “Ex vivo” (Latin: “out of the living”) means that the method takes place outside an organism and does not include any manipulation of the mammalian subject.

The term “diagnosis” when used herein means the process of determining which disease or condition a mammalian subject has or is afflicted with. With regard to the present invention it can, e.g., be detected if a mammalian subject has a cancer or suffers from a degenerative or inflammatory disease. Information required for diagnosis is typically obtained from the subject to be diagnosed in the form of a tissue sample. The tissue sample can be analyzed by MALDI imaging, a histochemical staining and FISH and, depending on the outcome, the subject is diagnosed to have the disease or not.

“Prognosis” as used herein relates to predicting the likely or expected development of a disease, including whether the signs and symptoms will improve or worsen (and how quickly) or remain stable over time; expectations of quality of life, such as the ability to carry out daily activities; the potential for complications and associated health issues; and the likelihood of survival including life expectancy. Prognosis may also relate to the stratification of the mammalian subject. “Stratification” as used herein describes the process of stratifying the mammalian subject into a group of mammalian subjects, e.g. whether the mammalian subject will benefit from a specific treatment or not.

A “disease” as used herein is a particular abnormal condition that negatively affects the structure or function of part or the entire mammalian subject. Diseases are often construed as medical conditions that are associated with specific symptoms and signs. The disease preferably manifests in the tissue, i.e. shows any abnormality that can be measured or detected using MALDI imaging, a histochemical staining and/or FISH. The in-vitro method of the present invention is suitable for the diagnosis of any disease that manifests in tissues. This includes, but is not limited to, cancer, a degenerative disease or an inflammatory disease. Furthermore, the disease preferably (i) alters any biomarker that can be detected by MALDI imaging and/or (ii) comprises a genetic and/or chromosomal abnormality. In a preferred embodiment, the disease is cancer. More preferably, the cancer is breast cancer, Barrett's cancer or gastroesophageal adenocarcinoma or all other types of cancer or their related precursor lesions.

The term “cancer” as used herein refers to proliferative diseases, such as lymphomas, lymphocytic leukemias, lung cancer, non-small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma and Ewings sarcoma, including refractory versions of any of the above cancers, or a combination of one or more of the above cancers.

A “tissue sample” as used herein relates to a sample obtained from the mammalian subject, e.g. a tissue sample that has been obtained from the mammalian subject by a biopsy. Samples may also include sections of tissues such as sections taken for histological purposes. “Tissue” as used herein relates to a cellular organizational level between cells and a complete organ. A tissue is an ensemble of similar cells and their extracellular matrix from the same origin that together carry out a specific function. Tissues of the present invention include, but are not limited to, epithelium, endothelium, mesothelium, mesenchyme, blood cells, neurons, germ cells, dermal tissue, vascular tissue, ground tissue or meristematic tissue. Preferably, the tissue samples are prepared for analysis by fixation. Fixation of tissues may include the application of e.g., formalin, glutaraldehyde or any other crosslinking reagent. After fixation, the tissues may be embedded in paraffin. Most preferably, the tissue sample is a formalin-fixed paraffin-embedded (FFPE) sample.

In case an FFPE sample is used, the tissue sample preferably is deparaffinized prior to step (i), the MALDI imaging analysis. Deparaffinization may relate to a removal of the paraffin that was used to protect the sample from degradation. An exemplary method for deparaffinization is described in Example 1. In short, the tissue sample is heated, e.g. to 70° C. for about 1 h and washed in a solvent such as ethanol, methanol or xylene, preferably xylene. Prior to use in the MALDI spectrometer, the tissue sample preferably is allowed to dry.

Within the context of the invention, the same tissue sample is used for every step (i), (ii) and (iii) of the in-vitro method of the invention. The “same sample” in this context means that a tissue sample is analyzed by MALDI imaging in step (i), the same tissue sample is analyzed by a histochemical stain in step (ii) and the same sample is also analyzed by FISH in step (iii). Importantly, the same sample does not only mean that different parts of the very same sample are analyzed but that the same area is analyzed by the methods of step (i) to (iii) to allow an overlay or co-registration of the information.

The term “mammalian subject” as used herein relates to any member of the class Mammalia. Members of the class of Mammalia include, but are not limited to, humans, rats, mice, dogs, cats, monkeys, or primates. Preferably, the mammalian subject is a human.

“Determining the level” of a biomarker or reference marker within the context of the invention relates to the detection of a biomarker. The result of the detection may be in the form of signal intensity or a yes or no answer. This level of the biomarker may then be compared to the level of the biomarker in another mammalian subject, preferably a healthy mammalian subject. Based on the biomarker, a deviation of the level, i.e. the signal intensity, may be indicative for a diagnosis of the disease or may be indicative for a good or bad prognosis. Determining of the level does not necessarily relate to absolute concentrations but must enable the comparison between the tissue sample of the mammalian subject to be tested and a tissue sample from a healthy or control mammalian subject. The determination of the level of the first biomarker is done by MALDI-imaging while the determination of the level of the second biomarker and the reference biomarker is performed by a FISH analysis.

MALDI imaging mass spectrometry (MALDI-IMS) or MALDI imaging is the use of matrix-assisted laser desorption ionization as a mass spectrometry imaging technique in which the sample, often a thin tissue section, is moved in two dimensions while the mass spectrum is recorded. MALDI imaging allows analyzing the spatial distribution of metabolites in a cell. The 2D spectrum may be then transformed into a heat map for each m/z species and correlated with an optical image of the sample. Preferably, the sample has been stained by a histochemical and/or immunohistochemical staining such as a hematoxylin & eosin staining before obtaining the optical image of the sample. The overlay of the MALDI imaging results, e.g. the 2D mass spectrum in form of a heat map for one m/z species, over the image of the sample then allows the precise spatial allocation of specific m/z species in the sample. This process can be also called “co-registration” and/or “image analysis”. For facilitation of this process, the sample is scanned to acquire tissue images for co-registration, and/or image analysis purposes prior to step (i).

MALDI preferably makes use of a MALDI matrix. A “MALDI matrix” typically comprises molecules that absorb the laser energy used for ionization. Typical MALDI matrix molecule include 2,5-dihydroxy benzoic acid, 3,5-dimethoxy-4-hydroxycinnamic acid, 4-hydroxy-3-methoxycinnamic acid, α-cyano-4-hydroxycinnamic acid, picolinic acid, 3-hydroxy picolinic acid or 9-aminoaciridine. A solution of one of these molecules may be made, often in a mixture of highly purified water and an organic solvent such as acetonitrile (ACN) or ethanol. A counter ion source such as Trifluoroacetic acid (TFA) may be added to generate the [M+H] ions. Preferably, the MALDI matrix is 9-aminoacridine.

Beside the usage of a MALDI matrix, chemical in situ derivatization approaches can be used to increase selectivity and/or sensitivity for the mass spectrometry analysis of compounds. The derivatization is used to transform a chemical compound into a product (the reactions' derivate) of similar chemical structure. Generally, a specific functional group of the compound participates in the derivatization reaction and transforms the educt to a derivate of deviating reactivity, solubility, boiling point, melting point, aggregate state, or chemical composition. Resulting new chemical properties are used for quantification or separation of the educt. A selective derivatization of analyte compounds can be used to enhance ionization and/or desorption yields for specific compounds detection.

In general, a “biomarker” may be a measurable indicator of the severity or presence of a disease. More generally a biomarker is anything that can be used as an indicator of a particular disease state or some other physiological state of an organism. Examples for biomarkers include proteins, lipids, nucleic acids such as DNA or RNA, hormones, metabolites or any other suitable compound.

A “tumor marker” as used herein relates to any biomarker that enables the diagnosis or prognosis of a tumor. Examples for a tumor marker include, but are not limited to, Alpha fetoprotein, CA15-3, CA27-29, CA19-9, CA-125, calcitonin, calretinin, carcinoembryonic antigen, CD34, CD99, CD117, chromogranin, cytokeratin, desmin, epithelia membrane antigen, CD31, glial fibrillary acidic protein, gross cystic disease fluid protein, HMB-45, human chorionic gonatogropin, HER2/neu, immunoglobulin, inhibin, keratin, MART-1, Myo D1, muscle-specific actin, neurofilament, neuron-specific enolase, placental alkaline phosphatase, prostate-specific antigen, PTPRC, S100 protein, smooth muscle actin, syntoptophysin, thymidine kinase, thyroglobulin, thyroid transcription factor-1 or vimentin.

The method of the present invention is not limited to the analysis of one first biomarker and one second biomarker. To further increase the diagnostic value of the method of the invention, further biomarkers may be analyzed. Accordingly, the method of the invention may further comprise the determination of the level of at least one additional biomarker, preferably the in-vitro method further comprises the determination of the level(s) of 1, 2, 3, 4, 5, 6, 7, 8, 9, 10 or more than 10 additional biomarkers in step (i). Further, the method of the invention may further comprise the determination of the level of at least one additional biomarker, preferably the in-vitro method further comprises the determination of the level(s) of 1, 2, 3, 4, 5, 6, 7, 8, 9, 10 or more than 10 additional biomarkers in step (iii).

“Histochemical staining” as used herein relates to the use of stains and dyes to highlight structures in tissue samples for viewing and analyzing. Stains may be used to define and examine bulk tissues (highlighting, for example, muscle fibers or connective tissue), cell populations (classifying different blood cells, for instance), or organelles within individual cells. More particularly, histochemical staining may involve adding a class-specific (DNA, proteins, lipids, carbohydrates) dye to a substrate to qualify or quantify the presence of a specific compound. Simple histochemical staining is staining with only one stain/dye. There are various kinds of multiple staining, many of which are examples of counterstaining, differential staining, or both, including double staining and triple staining. Such multiple stainings include, but are not limited to, the hematoxylin and eosin (H&E), and/or any combination of acridine orange, Bismarck brown, carmine, coomassie blue, cresyl violet, crystal violet, DAPI, ethidium bromide, acid fuchsine, Hoechst, iodine, malachite green, methyl green, methylene blue, neutral red, nile blue, nile red, osmium tetroxide, propidium iodide, rhodamine, oil red 0 or safranin staining. Preferably, the histochemical of the tissue sample is an H&E staining. In this staining, nuclei appear to be blue/purple, the cytoplasm appear to be red and collagen and mitochondria appear to be in pale pink. A person skilled in the art can readily realize whether a sample comprises abnormalities that would be indicative for a disease.

“Immunohistochemical staining” (IHC staining) as used herein relates to the process of selectively imaging antigens (proteins) in sample, such as a tissue sample or tissue section, by exploiting the principle of antibodies binding specifically to antigens in biological tissues. Immunohistochemical staining is widely used in the diagnosis of abnormal cells such as those found in cancerous tumors. Specific molecular markers are characteristic of particular cellular events such as proliferation or cell death (apoptosis). Visualizing an antibody-antigen interaction can be accomplished in a number of ways. In the most common instance, an antibody is conjugated to an enzyme, such as peroxidase, that can catalyze a color-producing reaction. Alternatively, the antibody can also be tagged to a fluorophore, such as fluorescein or rhodamine.

Step (ii) of the in-vitro method of the invention may further comprise also the digitalization, image analysis and/or morphometry. The digitalization (scanning) of the sample allows the automated analysis of the digital image of the sample. Morphometry relates to the quantitative analysis of a form of, e.g. a cell within the sample. Morphometry, when used within the context of the in-vitro method of the invention, may be used to analyze alterations or deviations caused by a disease. After performing the histochemical and/or immunohistochemical staining of step (ii) of the method of the invention, the sample preferably is washed.

“Determining the ratio” as used herein relates to the comparison of the levels of the second biomarker and the reference biomarker. When the level of a biomarker or reference marker is determined, the person skilled in the art can compare the signal intensities or any other value that can be determined of a biomarker, here e.g. the second biomarker, and the reference marker. The ratio the second biomarker and the reference marker may be altered in a mammalian subject that suffers from a disease in comparison to a healthy or control mammalian subject.

A “reference marker” however is not meant to be associated with the disease but preferably is unaltered by the presence or absence of the disease and thereby allows to normalize the biomarker to the reference marker. Examples for biomarkers include proteins, lipids, nucleic acids such as DNA or RNA, hormones, metabolites or any other suitable compound. Examples for reference markers include, but are not limited to, the centromeric region of chromosome 17 (CEP17), anaplastic lymphoma kinase (ALK), MET Exon 14 Skipping Mutations (MET), E3 ubiquitin-protein ligase (MDM2), Proto-oncogene tyrosine-protein kinase ROS (ROS1), RET proto-oncogene (RET), Platelet-derived growth factor subunit B (PDGFB), N-myc proto-oncogene (NMYC), epidermal growth factor receptor (EGFR), 1p/1q-FISH and 19p/19q-FISH, Aneuvysion-Multicolor-FISH, BCR-ABL-FISH, C-MYC-breakpoint-FISH, Ewing sarcoma breakpoint region 1-FISH, IGH-breakpoint-FISH, LAVysion-Multicolor-FISH, Light chains in situ hybridization, Melanom-Multicolor-FISH, SYT-breakpoint-FISH, t(11;14)(q13;q32)-FISH (mantle cell lymphoma), t(11;18)(q21;q21)-FISH (lymphoma-associated translocation 1), t(14;18)(q32;q21)-FISH (follicle centre lymphoma), TP53/CEN7-FISH, Urovysion-Multicolor-FISH.

“FISH” as used herein relates to a molecular cytogenetic technique that uses fluorescent probes that bind to only those parts of the chromosome with a high degree of sequence complementarity. It may be used to detect and localize the presence or absence of specific DNA sequences on chromosomes. Fluorescence microscopy can be used to find out where the fluorescent probe is bound to the chromosomes. FISH may be used for finding specific features in DNA for use in genetic counseling, medicine, and species identification. FISH can also be used to detect and localize specific RNA targets (mRNA, IncRNA and miRNA) in samples such as cells, circulating tumor cells, and tissue section. In this context, it can help define the spatial-temporal patterns of gene expression within cells and tissues. This pattern of gene expression allows a person skilled in the art to diagnose a disease in the sample.

“Suspicious for the disease” as used herein relates to a region of the sample, which shows any alteration in comparison to a sample of a healthy or control mammalian subject. A person skilled in the art is aware how to interpret stainings of samples such as the stainings performed in step (ii) in the in vitro method of the invention. For example, a cell or a region in a tissue sample shows any alteration after an H&E staining. In this case, the results of the MALDI imaging should be observed at this region that is suspicious for the disease.

### DETAILED DESCRIPTION

The present invention is described in detail in the following and will also be further illustrated by the appended examples and figures.

The inventors surprisingly found that the very same tissue sample can be used for MALDI imaging and subsequently be analyzed by fluorescence in situ hybridization (FISH). This finding is surprising given the fact that applying the MALDI procedure to the sample necessarily comprises the use of UV bombardment. Thus, a person skilled in the art would not have combined MALDI-imaging with FISH for analyzing tissue sections. The inventors showed for the first time the direct correlation of findings from MALDI imaging experiments and histological staining with the outcome of FISH analysis on the very same tissue section. This finding is very useful for the diagnosis of many diseases and allows the combination MALDI imaging, which allows the precise analysis of the cellular components such as metabolites in specific regions or even single cells that are suspicious for the disease as found by a staining, with a FISH analysis of that very same region or single cell. This provides an improved tool for diagnosis and prognosis of diseases.

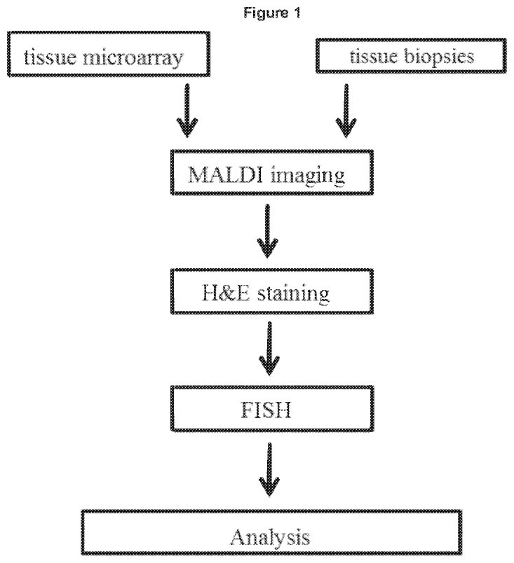

Thus, the method of the present invention represents a new in vitro method for tissue sample analytics, which allows molecular feature extraction by mass spectrometry imaging, and phenotypic and cytogenetic feature extraction by digital image analysis, resulting in highly improved diagnosis and/or prognosis of a disease. Thus, the overall information which can be gained from one tissue section is extended, and, in combination with clinical data including survival and response, this combined method may provide an improved method of diagnosis of diseases. An exemplary workflow of the in-vitro method of the present invention on a tissue sample is depicted in FIG. 1 and will be explained below:

The in-vitro method of the invention comprises the steps of:
(i) Determining the level of a first biomarker using matrix-assisted laser desorption/ionization (MALDI)-imaging in the sample;
(ii) Performing a histochemical staining of the sample including digitalization;
(iii) Determining the ratio of the levels of a second biomarker and a reference marker using fluorescence in situ-hybridization (FISH);
wherein the same tissue sample is used in steps (i) to (iii),
wherein the level of the first biomarker is analyzed in regions of the sample, which are suspicious for the disease as found by staining of step (ii), and wherein a deviation of the level of the first biomarker, and a deviation of the ratio of the second biomarker and the reference marker in comparison to a tissue sample of a healthy subject is indicative for the presence of the disease.

In general, step (i), the MALDI imaging, is the first method that is applied to the tissue sample. The order of steps (ii) and (iii) may however be changed, so that e.g. the following orders of steps are possible: (i), (ii) and (iii) or (i), (iii) and (ii). However, this does not necessarily mean that other technologies cannot be used before or after any of steps (i) to (iii). E.g., an immunohistochemical staining could be applied before or after the histochemical staining.

Before applying MALDI imaging that is used to determine the level of a first biomarker, the tissue sample may be prepared for MALDI imaging. The preparation may include the deparaffinization of the tissue sample, in case the tissue sample is an FFPE sample. Additionally, a MALDI matrix suitable for MALDI imaging is preferably applied prior to the actual MALDI imaging procedure. An exemplary MALDI matrix comprises 9-aminoacridine hydrochloride monohydrate. Before starting the MALDI imaging, the tissue sample may also be scanned or photographed to allow an easier allocation of the 2D mass spectrometry results.

Following the preparations of the tissue sample for the MALDI imaging procedure, the MALDI imaging of the tissue sample is carried out. As outlined herein, MALDI imaging is used to detect a first biomarker. The first biomarker preferably is a cellular component whose (local) concentration is altered in comparison to a healthy cell and/or a sample of a healthy human. Thus, a deviation of the level of the first biomarker may be indicative for the presence of a disease. Such a first biomarker may be a metabolite that is frequently accumulated or depleted in a human suffering from a disease. Suitable first biomarkers include, but are not limited to cAMP, ADP, Glycans, Pyruvate, Hexose-phosphate, N-Acetylhexosamine sulphate, Carnitine, dUMP, UDP, GMP, GDP, Estrone sulphate, Estradiol sulphate, Pregnenolone sulphate, Epinephrine, Cholesterole sulphate, DHEA sulphate, Hydroxyestrone, Oxocortisol, Hydroxycortisol, Oxalsuccinate, Citrate, Serine, Methionine, Tryptophane, Farnesyl diphosphate, N-Acetylneuraminate, or Phosphatidylinositol.

As outlined herein, it is an advantage of MALDI imaging that the level of the first biomarker may be measured in specific regions or even single cells. Thus, the determination of the level of the first biomarker may be concentrated on regions of the sample, which are suspicious for the disease as found by a staining such as the staining of step (ii). This high local resolution of the tissue sample enables to analyze only the regions of the sample, which are suspicious for the disease as found by the staining of step (ii). By concentrating on suspicious regions, the measurement of the first biomarker is restricted to the regions that may be altered by the disease. Thereby, the result is not influenced or “diluted” by parts of the sample that are not affected by the disease. Accordingly, the level of the first biomarker is preferably determined in regions of the tissue sample, which are suspicious for the disease as found by the staining of step (ii).

Mass spectrometry in general and time of flight (TOF)-mass spectrometry in particular, can be applied in two modes: positive ion mode and negative ion mode. “Positive” and “negative” in this context relates to polarity of the ions that are analyzed. In a negative ion mode, negatively charged ions are analyzed and vice versa, in a positive ion mode, positively charged ions are analyzed. In one embodiment, the MALDI imaging is performed in positive ion mode. In a more preferred embodiment, MALDI imaging is performed in negative ion mode.

MALDI is suitable for the analysis of ions of different masses. The mass range m/z may be in the range of 1-1000000, preferably in the range of 10-50000, more preferably in the range of 50-25000, even more preferably in the range of 50-15000 and most preferably in the range of 50-1000.

After the MALDI imaging procedure, either the histochemical staining or the FISH analysis of the same tissue sample may follow.

Prior to the histochemical staining or the FISH analysis, the MALDI matrix may be removed or washed away. The MALDI matrix may be removed by a suitable solvent that dissolves the matrix but does not alter the sample. In one embodiment, a solution comprising 50-90% ethanol, preferably comprising about 70% ethanol, is used for washing away and/or removing the MALDI matrix. In an alternate preferred embodiment, a solution comprising 96% xylol, 3% isopropanol and 1% methanol is used for removing the MALDI matrix.

The histochemical staining may, as outlined herein, be used to identify regions that are suspicious for a disease. Identifying such regions already implies that also the histochemical and/or immunohistochemical staining may provide further information that can be used for the diagnosis of the disease. This information, when combined with the results of the MALDI imaging and FISH, further increases the diagnostic value. The histochemical staining is preferably carried out directly after the MALDI imaging. However, it is also envisioned that the FISH analysis of the tissue sample can be carried out after the MALDI imaging and the immunohistochemical staining is carried out as 3^{rd} step.

In addition to the histochemical staining, an immunohistochemical staining may also be applied. Markers that may be analyzed include, but are not limited to, HER2/neu, EGFR, PD-L1. It is within the knowledge of a person skilled in the art to perform an immunohistochemical staining.

Again, after the histochemical staining, the same tissue sample can be washed and prepared for the FISH analysis.

The FISH analysis may be carried out as 3^{rd} step of the in-vitro method of the present invention or also as 2^{nd} step of the in-vitro method of the present invention. FISH is used to determine the ratio of the levels of a second biomarker and a reference biomarker. Since FISH is mainly used for cytogenetic analysis, the second biomarker is preferably a cytogenetic marker. Cytogenetic markers include genes or whole chromosomes, whose number is altered. By comparison to a reference marker that is not altered by the disease, a ratio can be determined that is indicative for the presence of the disease. Examples for biomarkers suitable as second biomarkers include, but are not limited to, HER2/neu, anaplastic lymphoma kinase (ALK), MET Exon 14 Skipping Mutations (MET), E3 ubiquitin-protein ligase (MDM2), Proto-oncogene tyrosine-protein kinase ROS (ROS1), RET proto-oncogene (RET), Platelet-derived growth factor subunit B (PDGFB), N-myc proto-oncogene (NMYC), epidermal growth factor receptor (EGFR), 1p/1q-FISH and 19p/19q-FISH, Aneuvysion-Multicolor-FISH, BCR-ABL-FISH, C-MYC-breakpoint-FISH, Ewing sarcoma breakpoint region 1-FISH, IGH-breakpoint-FISH, LAVysion-Multicolor-FISH, Light chains in situ hybridization, Melanom-Multicolor-FISH, SYT-breakpoint-FISH, t(11;14)(q13;q32)-FISH (mantle cell lymphoma), t(11;18)(q21;q21)-FISH (lymphoma-associated translocation 1), t(14;18)(q32;q21)-FISH (follicle centre lymphoma), TP53/CEN7-FISH, or Urovysion-Multicolor-FISH.

In a preferred embodiment, the second biomarker may be HER2/neu and the reference biomarker may be the centromeric region of the chromosome 17 (CEP17). This combination allows the diagnosis of cancer, in particular the diagnosis of HER2/neu-positive cancers.

After performing these at least three measurements—MALDI imaging, histochemical staining and FISH analysis—, the results are be integrated to allow the diagnosis and/or prognosis of the disease. Integration in this context means the combination of the single results of each single step, the MALDI imaging, the FISH analysis and information of histochemical and optionally also immunohistochemical stainings. While one of these results does not have to provide a clear result, the combination of the individual results will improve the diagnostic value of the in-vitro method of the present invention as a whole.

A more detailed, but non-limiting and only exemplary workflow for the diagnosis of HRE2/neu-positive gastroesophageal adenoma carcinoma is shown in Example 1. Here, the inventors combined MALDI imaging for AMP as first biomarker and the ratio of HER2/neu as second biomarker and CEP17 as reference biomarker.

State of the art in HER2/neu diagnosis is immunohistochemical staining and in situ hybridization (Lordick, 2017). The immunohistochemical staining, directly labelling HER2/neu, is scored as 0, 1+, 2+, or 3+. Score 0 and 1+ are defined as HER2/neu negative, which means anti-HER2/neu treatment is not considered at all, and 3+ is HER2/neu positive, an outcome, which recommends anti-HER2/neu treatment. The score 2+ represents an intermediate state which requires further testing. In this case in situ hybridization is performed as fluorescence or chromogenic in situ hybridization. Hereby, the signals of labelled gene copies are counted in at least 20 tumor cell nuclei and a ratio of gene copy number and chromosome number is calculated and defined as amplified when the ratio is 2. Samples with a ratio of 2 or more is then also defined as HER2/neu positive and leads to an HER2/neu directed treatment (Lordick 2017, Rauser 2007, Bartley 2017). In HER2/neu testing, FISH is an FDA approved method for the enumeration of the absolute HER2/neu gene copy number in breast cancer and gastroesophageal adenocarcinoma.

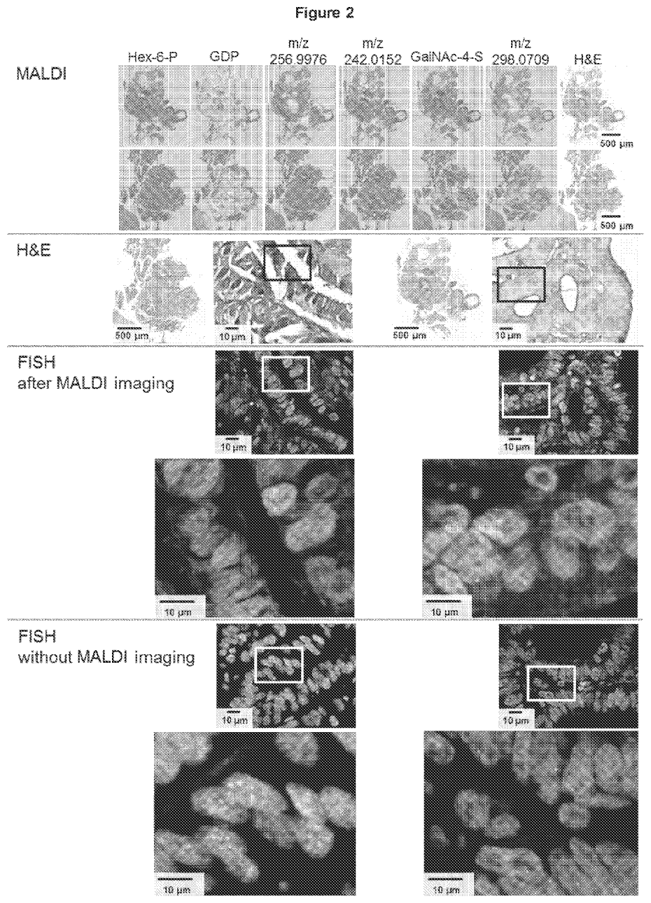

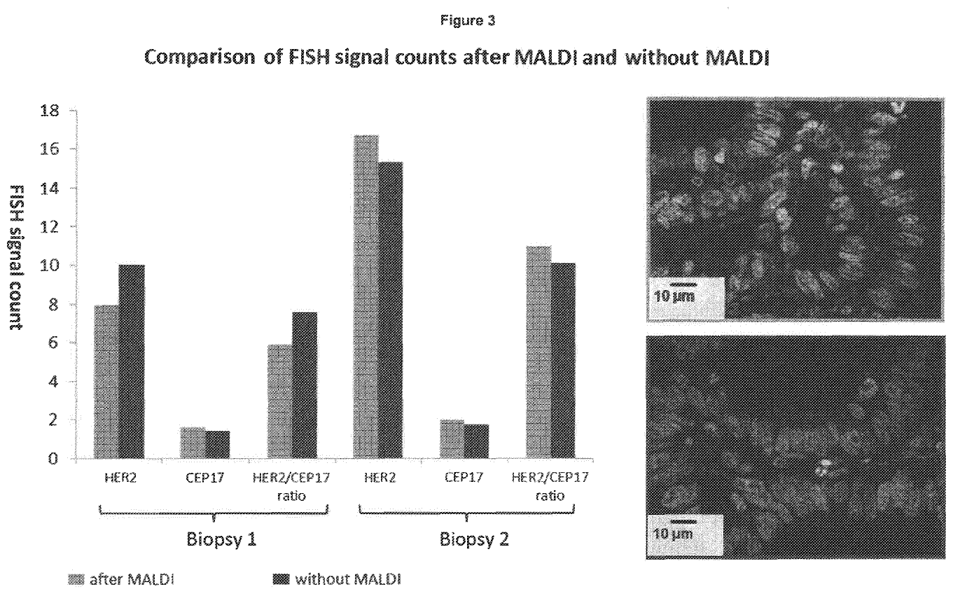

The exemplary approach of the inventors was established and validated using human gastroesophageal adenocarcinoma biopsies. A comparison of a FISH analysis after MALDI imaging with a reference FISH analysis without previous MALDI imaging using consecutive sections of the same biopsies was performed (FIGS. 2 and 3). The results from both approaches revealed equal results. Most noticeably, after the performance of the whole protocol, the cell structure is still preserved and FISH results remain as correct and reliable as when performed exclusively. The inventors demonstrated that their protocol does not only keep the tissue structure intact, but, most remarkably, it even leaves the DNA structure unimpaired and thus the FISH approach remains unaffected by the foregoing procedure. Thus, the method of the invention, in particular the MALDI imaging protocol, preferably does not damage the sample and does not influence the outcome of subsequent FISH experiments.

As shown in Example 1, HER2/neu gene amplification and AMP mass intensity both individually have the ability to differ significantly between good and poor prognosis group in the Kaplan-Meier analysis, while not correlating to each other. Using this ratio, it was possible to increase the significance level of the survival analysis to p=0.000002875. Consequently, the information from the gene amplification analysis and the mass spectrometry imaging approach together provide a very precise prognosis and thereby show a synergistic effect of the combination of these methods.

Accordingly, in one embodiment, the disease is cancer, the first biomarker is AMP, wherein an increased AMP level is indicative for a diagnosis of cancer. Alternatively or additionally, the disease is cancer and the second biomarker is HER2/neu (locus 17q11.2-q12) and the reference marker is the centromeric region of chromosome 17 (CEP17; locus 17p11.1-q11.1), wherein the disease is cancer and a ratio of HER2/neu to CEP17 of 2 or higher is indicative for a diagnosis of cancer.

In this method, the first biomarker is adenosine monophosphate (AMP), the staining in step (ii) is hematoxylin and eosin stain (H&E stain), the second biomarker is HER2/neu and the reference marker is CEP17 and wherein an increase of the ratio of AMP to the ratio of HER2/neu and CEP17 is indicative for the presence of a cancer.

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “less than” or in turn “more than” does not include the concrete number.

For example, less than 20 means less than the number indicated. Similarly, more than or greater than means more than or greater than the indicated number, e.g. more than 80% means more than or greater than the indicated number of 80%.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”. When used herein “consisting of” excludes any element, step, or ingredient not specified.

The term “including” means “including but not limited to”. “Including” and “including but not limited to” are used interchangeably.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications cited throughout the text of this specification (including all patents, patent application, scientific publications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

### EXAMPLES

An even better understanding of the present invention and of its advantages will be evident from the following examples, offered for illustrative purposes only. The examples are not intended to limit the scope of the present invention in any way.

### Example 1: Diagnosis of Gastroesophageal Adenocarcinoma

### Material and Methods

### Human Tissue Samples

71 human gastroesophageal adenocarcinoma patient samples were analyzed.

Tissue samples were fixed for 12-24 hours in 10% neutral buffered formalin and then embedded in paraffin using standardized automated procedures. Prior to embedding in molten paraffin, the samples were dehydrated in increasing ethanol series and cleared using xylene. The resulting paraffin blocks were then used for the preparation of a tissue microarray (TMA).

2 biopsies of human gastroesophageal adenocarcinoma were chosen for the establishment and validation of the workflow.

### Overview of the Workflow

The example is based on the combination of two different modalities: measuring metabolites by MALDI imaging and gene copy number by fluorescence in situ hybridization (FISH) using one very same formalin-fixed and paraffin-embedded (FFPE) tissue section for all imaging steps. The workflow used in this example is depicted in FIG. 1, starting with the MALDI imaging measurement followed by H&E staining. For performing the second modality, FISH, the cover glass was removed and a washing procedure was carried out to ensure the complete elimination of mounting medium from the tissue. As a last step, data gained from MALDI imaging and FISH were fused in order to enhance prognosis in the patient cohort (FIG. 1).

### Tissue Preparation

4 μm thick tissue sections were cut using a CM1950 cryostat (Leica Microsystems, Wetzlar, Germany) and mounted onto indium-tin-oxide (ITO) coated glass slides (Bruker Daltonik GmbH, Bremen, Germany), which were previously covered with 1:1 poly-L-lysine (Sigma-Aldrich; Taufkirchen, Germany) and 0.1% Nonidet P-40 (Sigma-Aldrich; Taufkirchen, Germany). For deparaffinization, sections were incubated at 70° C. for 1 hour and washed twice in xylene for 8 min. Prior MALDI matrix application, tissue sections were air-dried on a heating plate at 35° C. for 1 min and scanned using a flatbed scanner in order to acquire digital tissue images for co-registration purposes. Subsequently, slides were coated with 10 mg/ml 9-aminoacridine (Sigma-Aldrich; Taufkirchen, Germany) in 70% methanol as matrix solution using a SunCollect spraying device (Sunchrom, Friedrichsdorf, Germany).

### MALDI Imaging

MALDI imaging was performed using a Solarix 7 T FT-ICR mass spectrometer (Bruker Daltonik GmbH, Bremen, Germany) at a spatial resolution of 60 pm in negative ion mode in the mass range of m/z 50-1000, whereby 50 laser shots were accumulated for each position measured. The software packages FlexImaging 4.0 and SolarixControl 3.0 (Bruker Daltonik GmbH, Bremen, Germany) were applied for data generation and visualization as previously described (Ly 2016, Kunzke 2017).

After MALDI measurement, the matrix was removed by a washing step in 70% ethanol for 1 min and was subsequently stained with histological hematoxylin and eosin staining as described previously. The Cover glass was mounted using Pertex mounting medium (Medite GmbH, Burgdorf, Germany).

For digitalization, slides were scanned at 20× objective magnification with a slide scanner (Mirax Desk, Carl Zeiss Microlmaging GmbH, Jena, Germany). For co-registration with MALDI imaging data, the images were imported into the Flexlmaging 4.0 software (Bruker Daltonik GmbH, Bremen, Germany).

### FISH Experiment

The FISH experiment was performed using the tissue sections which were stained with hematoxylin and eosin after the MALDI imaging measurement (see also the workflow in FIG. 1).

The Slides were incubated in xylene at room temperature for 12 hours before the cover glass was removed. Further washing steps in xylene and isopropyl alcohol, each for the duration of 1 hour, were carried out subsequently. This washing procedure was followed by a series of decreasing ethanol concentrations from 100% down to 50%, whereas the sections were immersed for 5 min at each step before they were transferred into demineralized water.

After incubating for 5 min in PBS (Sigma-Aldrich; Taufkirchen, Germany) at room temperature, the sections were boiled in citrate buffer containing 0.1 M citric acid (Sigma-Aldrich; Taufkirchen, Germany) and 0.1 M sodium citrate (Sigma-Aldrich; Taufkirchen, Germany) for 20 min using a microwave oven at 350 W. Afterwards, the sections were washed in PBS and incubated in Pronase E 0.05% (Sigma-Aldrich; Taufkirchen, Germany) for 5 min at 37° C. Again, one washing step in PBS was performed before the sections were dehydrated in ascending alcohol series, 5 min in each concentration, at −20° C. The sections were then air-dried at room temperature and heated on a heat plate at 37° C. for 1 min.

HER2/neu-CEP 17 probes (PathVysion HER2 DNA Probe Kit II, Abbott, Illinois, USA) were added to the slide, still placed on a 37° C. heat plate, covered with Fixogum rubber cement (Marabu) and stored in the dark. Denaturation happened simultaneously by increasing the temperature of the heat plate to 75° C. for 8 min. For hybridization, slides were kept in a humid atmosphere at 37° C. for 16 hours. After the incubation, slides were washed by short immersion in 2× SSC (Sigma-Aldrich; Taufkirchen, Germany) containing 0.3% Nonidet P-40 at room temperature and for 2 min in 2×SSC containing 0.3% Nonidet P-40 at 73° C. After air-drying, slides were stained using Hoechst (Sigma-Aldrich; Taufkirchen, Germany) at room temperature and air-dried again. Cover glasses were mounted using Vectashield mounting medium (Biozol, Eching, Germany). The kit consists of directly labeled, fluorescent DNA probes specific for the HER2/neu gene locus (17q11.2-q12) and a DNA probe specific for the a satellite DNA sequence at the centromeric region of chromosome 17 (17p11.1-q11.1).

FISH analysis of the biopsy sections without previous MALDI imaging was performed in equal manner.

Evaluation of the FISH experiment included counting of the fluorescent labels for gene copy numbers and centromeric region. Therefore a Z1 ZEISS Axioimager microscope (Zeiss, Jena, Germany) with a 63× magnification water objective was used.

### Data Analysis

For processing of the MALDI imaging data, a MATLAB script using the bioinformatics and image processing toolboxes (MathWorks, Natick, Mass., USA) was employed. Spectra which were exported by the Flexlmaging 4.0 software (Bruker Daltonik GmbH, Bremen, Germany) underwent baseline subtraction, resampling and smoothing as described previously (Buck 2017, Kunzke 2017). A signal-to-noise threshold of 2 was used and isotope peaks were excluded automatically. HMDB database was employed for the identification of m/z species with a mass tolerance of 3 ppm. The resulting peak intensity of AMP was exported to Microsoft Excel for data fusion with the FISH results.

The HER2/neu FISH approach was evaluated according to the recent guidelines (Bartley 2017). The signals for HER2 gene loci and CEP 17 centromere of 20 non-overlapping tumor cell nuclei were counted manually. The ratio HER2/CEP 17 was calculated using Excel, whereas a HER2/CEP 17 ratio ≥2.0 was considered as HER2/neu amplification (Bartley 2017). Furthermore, we differentiated the non-amplified samples. A HER2/CEP 17 ratio ≥1.1 and <2 was classified as low-level copy number gain (Rauser 2007).

Kaplan-Meier survival tests were performed using R statistics software and Prism was used for correlation plotting.

### Results

### MALDI Imaging Leaves Nuclear Morphology of Formalin-Fixed and Paraffin-Embedded (FFPE) Tissue Intact.

Human gastroesophageal adenocarcinoma biopsies, in the form of formalin-fixed and paraffin-embedded (FFPE) tissues, were used for validation of the fluorescence in situ hybridization (FISH) analysis either following MALDI imaging measurement or without a previous MALDI imaging measurement. One section of each biopsy sample was processed for MALDI FT-ICR metabolite imaging. Molecule visualizations resulting from the MALDI imaging approach are displayed in FIG. 2. The molecule signals follow the tissue morphology and, after co-registration with the H&E staining, it is possible to precisely allocate mass signals with tissue structures (FIG. 2). The overlay of MALDI imaging and H&E enables the identification and evaluation of specific tissue structures, e. g. tumor cell regions.

After analysis of the combined MALDI imaging, the cover glass was removed and FISH analysis was performed in order to detect HER2/neu gene amplification (see also the workflow exemplified in FIG. 1). FIG. 2 shows the direct allocation of tissue structures, precisely identifiable in the H&E staining, with the fluorescence microscopy image of the FISH analysis. In FISH analysis, nuclei are the only cell components which are stained (grey) (FIG. 2). The HER2/neu gene loci and the centromere region of chromosome 17 are fluorescent labelled by hybridization with red fluorescent probe (HER2/neu) and green fluorescent probe (CEP17), which enables counting of signal numbers and thereby calculating the HER2/CEP 17 signal ratio. There are 2 major preconditions for reliable HER2/neu testing: the nuclei must be stained clearly in order to allocate all signals belonging to each nucleus and fluorescence signals for both, HER2/neu probe and centromere probe CEP17, have to be clearly visible to enable distinct recognition of single signals.

The FISH analysis was evaluable even after the MALDI imaging procedure. Single fluorescence signals, even in non-amplified tissues were clearly visible and enumeration was possible just as in tissues which were not used for the MALDI imaging procedure. The nuclear morphology was clear and completely unaffected by MALDI imaging. The Hoechst nuclear staining allowed the detection of tumor areas and even cytomorphological details of the nuclear structure remained unchanged after MALDI imaging.

The ratios of HER2/neu gene locus (red) and centromeric region of chromosome 17 (green) signals were calculated, whereas a ratio 2.0 was designated as HER2 amplification (Rauser 2007). In the FISH experiment we found high-level amplification of HER2/neu gene copy number in both biopsies (FIG. 2). The FISH experiment which was performed after MALDI imaging and H&E staining resulted in excellent signals which allowed a very precise detection of gene amplifications (FIG. 2).

HER2/neu testing by FISH after MALDI and without MALDI reveals equal results

In order to validate the findings from the samples, which underwent the MALDI imaging procedure, consecutive reference sections of both biopsies were analyzed by FISH as a reference without MALDI imaging and H&E staining (FIG. 2, lowest panel). The FISH experiment was evaluated identically for the sections, which underwent the MALDI imaging protocol, as for those reference sections without previous MALDI imaging procedure. As shown in FIG. 2, both sections from both biopsies showed HER2/neu amplification. The quantitative evaluation of both approaches is shown in FIG. 3. FISH signals from 20 tumor cell nuclei were enumerated manually in biopsies which were analyzed by FISH after the MALDI imaging procedure or without previous MALDI imaging, respectively. Signal counts of HER2/neu and CEP17 of both biopsies were compared after the MALDI workflow and without previous MALDI and the HER2/CEP17 signal ratio was calculated. For biopsy 1, an average of 7.95 HER2/neu signals was detected after MALDI and 10.00 signals without MALDI workflow. CEP17 signal count revealed 1.60 and 1.45 signals per nucleus, respectively. Biopsy 2 showed HER2/neu amplification with a mean HER2/neu signal count of 16.75 after MALDI and 15.35 without MALDI. With a CEP17 number of 2.00 and 1.75, the resulting HER2/CEP17 signal ratios were 11.03 and 10.12.

Comparing the quality of the tissue after the combined workflow with the tissue that underwent only the FISH experiment, there is surprisingly no difference in quality and evaluability.

### HER2/Neu Testing After MALDI Imaging Allows Accurate Disease Prognosis

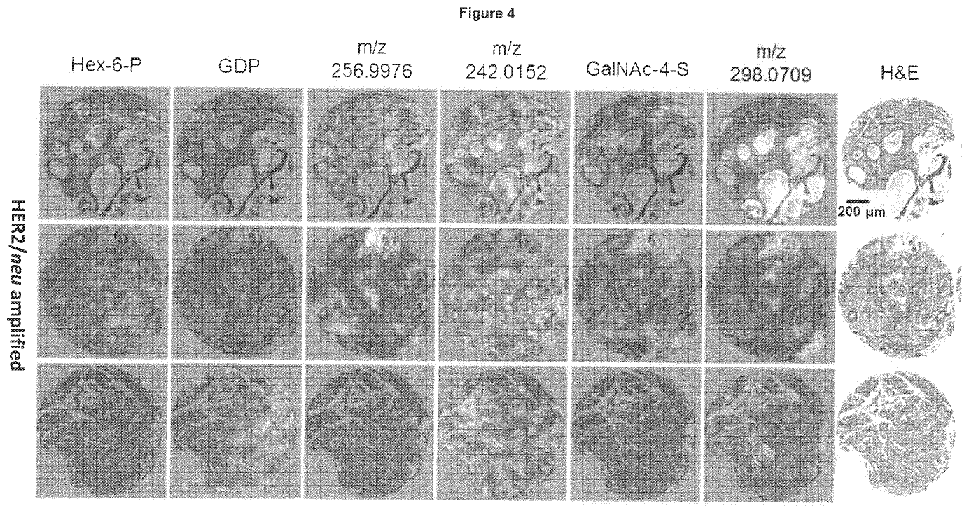

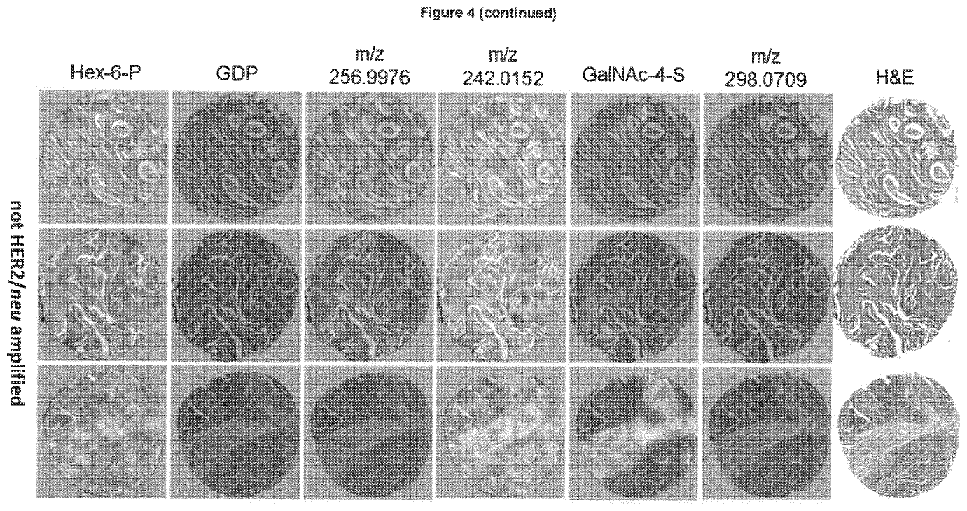

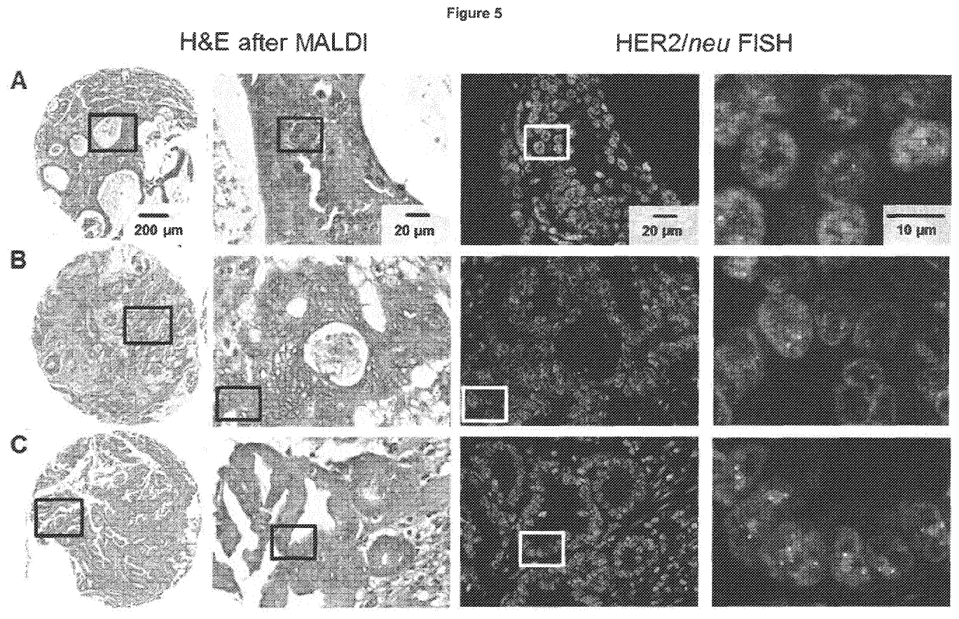

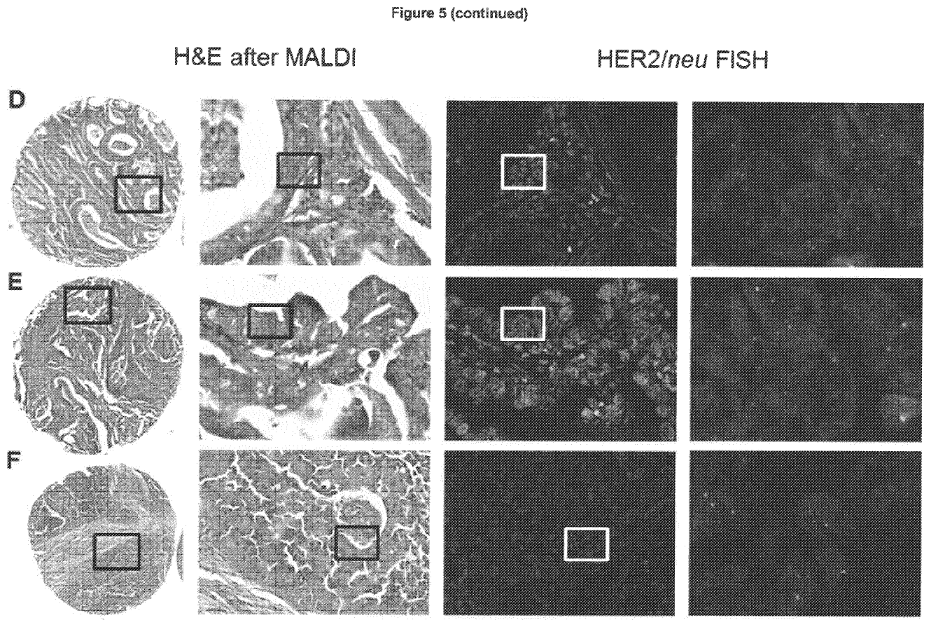

A tissue microarray (TMA) containing 69 human gastroesophageal adenocarcinoma patient samples was analyzed using the established pipeline of MALDI imaging followed by FISH. FIG. 4 displays the distribution of several molecules measured by MALDI imaging. As established for the biopsies, the TMA underwent FISH after the MALDI imaging procedure following the same workflow as described above. The results from the FISH analysis are presented in FIG. 5. Tissue cores in FIGS. 5A and B showed low/medium level HER2 amplifications, while the core in FIG. 5C was a highly amplified sample. The samples in FIGS. 5D, E, and F were found to be not HER2/neu amplified.

In sum, 9 tissue cores were found to be HER2/neu amplified, while 60 were not amplified. In the group of non-amplified cores, 18 were found to show low-level copy number gain. Average HER2/neu signal counts varied from 0.78 to 15.41, the average HER2/neu signal count of all observed cases was 2.44 signals per nucleus.

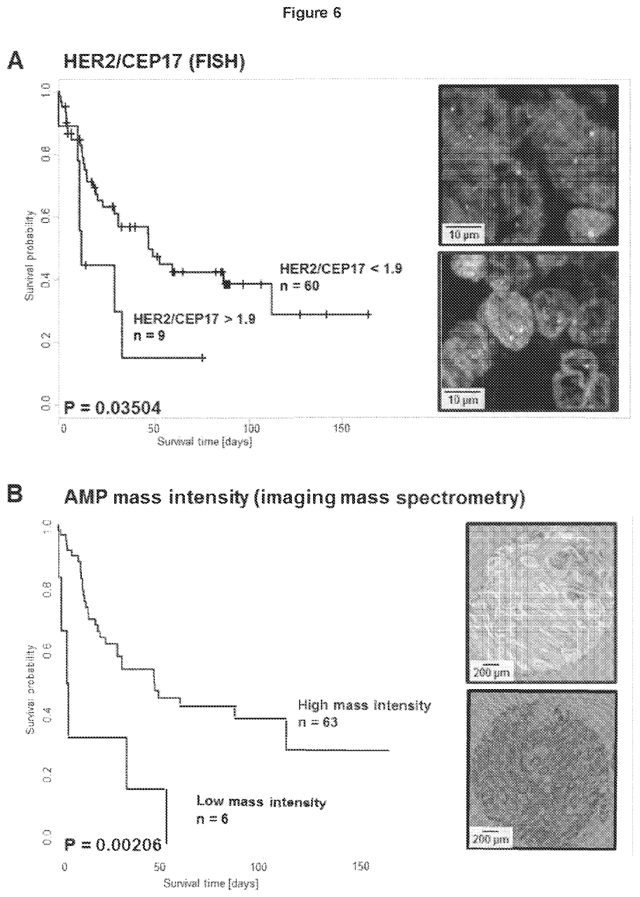

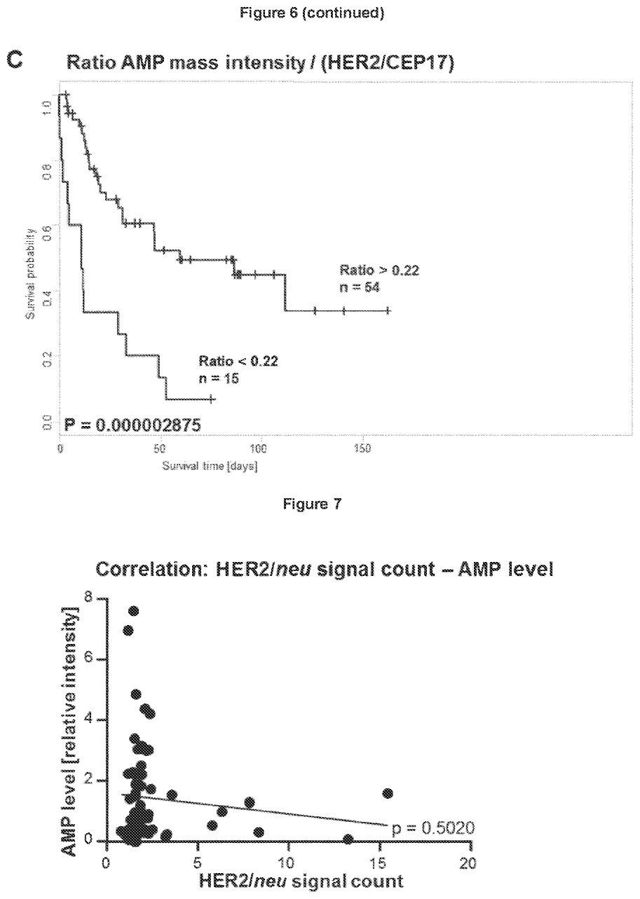

The FISH evaluation of the tissue microarray was analyzed statistically using the Kaplan-Meier survival test (FIG. 6). Hereby a significant (p=0.0350) difference in patient survival was found, outlining HER2/CEP17 signal ratio as a marker for patient survival (FIG. 6A).

### Adenosine Monophosphate Measured by MALDI Imaging Enables Survival Analysis

In the MALDI imaging approach, the H&E staining was used for the determination of tumor regions. Thus it was possible to extract mass spectra specifically from the tumor areas for analysis. The inventors focused on the peak intensity of adenosine monophosphate (AMP, m/z 346.0570). AMP signal intensity allowed significant prediction of patient survival (p=0.00206). Hereby the mass intensity was found to be higher in the good prognosis group, while signal intensity was weak in the poor prognosis group (FIG. 6B). In general, average AMP peak intensities varied between 0 and 7.59. In 39 cases the mean AMP intensity was found to be below 1.0 and 2 cases showed an intensity of more than 5. The intensity of 28 cases was in the medium range between 1.0 and 5.0. The overall average AMP intensity is 1.42. In the samples which were stratified as poor survivors (FIG. 5B), AMP signal intensity was below 0.15.

### Integration of FISH and MALDI Parameters Improves Prognosis

The inventors combined the data revealed from both approaches, AMP signal intensity by MALDI imaging and gene amplification by FISH, using the ratio of the mass intensity and the HER2/CEP17 signal ratio. This lead to an increase of the significance of the survival analysis with p=0.000002875. The patients in the good prognosis group showed a higher AMP/FISH ratio than the patients in the poor prognosis group. The calculated threshold for stratification of patient survival was 0.22 (FIG. 6C).

In FIG. 7, AMP signal intensities from MALDI imaging were plotted against the HER2/CEP17 signal ratio revealed from FISH in order to detect whether there is a correlation of the abundance of both features. Each data point represents a single patient. The random distribution of the data points in the plot depict the fact that both parameters do not correlate with each other (p=0.5020).

### REFERENCES

Bartley A, Washington M, Colasacco C, et al. HER2 Testing and Clinical Decision Making in Gastroesophageal Adenocarcinoma: Guideline From the College of American Pathologists, American Society for Clinical Pathology, and the American Society of Clinical Oncology. J Clin Oncol 2017; 35(4):446-464.
Buck A, Aichler M, Huber K, et al. In Situ Metabolomics in Cancer by Mass Spectrometry Imaging. Adv Cancer Res 2017; 134:117-132.
Kunzke T, Balluff B, Feuchtinger A, et al. Native glycan fragments detected by MALDI-FT-ICR mass spectrometry imaging impact gastric cancer biology and patient outcome. Oncotarget. 2017; 8(40):68012-68025.
Lordick F, Al-Batran S E, Dietel M, et al. HER2 testing in gastric cancer: results of a German expert meeting. J Cancer Res Clin Oncol 2017; 143(5):835-841.
Ly A, Buck A, Balluff B, et al. High-mass-resolution MALDI mass spectrometry imaging of metabolites from formalin-fixed paraffin-embedded tissue. Nat Protoc. 2016; 11(8):1428-43.
Rauser S, Weis R, Braselmann H, Feith M, Stein H J, Langer R, Hutzler P, Hausmann M, Lassmann S, Siewert J R, Hofler H, Werner M, Walch A. Significance of HER2 low-level copy gain in Barrett's cancer: implications for fluorescence in situ hybridization testing in tissues. Clin Cancer Res. 2007 Sep. 1; 13(17):5115-23.

1. An in-vitro method for diagnosis and/or prognosis of disease in a formalin-fixed paraffin-embedded (FFPE) tissue sample obtained from a mammalian subject comprising
(i) Determining the level of a first biomarker using matrix-assisted laser desorption/ionization (MALDI)-imaging in the sample;
(ii) Performing a histochemical staining of the sample including digitalization;
(iii) Determining the ratio of the levels of a second biomarker and a reference marker using fluorescence in situ-hybridization (FISH);
wherein the same tissue sample is used in steps (i) to (iii),
wherein the level of the first biomarker is analyzed in regions of the sample, which are suspicious for the disease as found by staining of step (ii), and wherein a deviation of the level of the first biomarker, and a deviation of the ratio of the second biomarker and the reference marker in comparison to a tissue sample of a healthy subject is indicative for the presence of the disease.

2. The in-vitro method of claim 1, wherein the sample is deparaffinized prior to step (i).

3. The in-vitro method of any one of claim 1 or 23, wherein the sample has been coated with a MALDI matrix prior to step (i) preferably after deparaffinization, wherein the MALDI matrix comprises 9-aminoacridine hydrochloride monohydrate.

4. The in-vitro method of any one of claims 1-3, wherein the sample has been coated with chemical substances for derivatization prior to step (i) preferably after deparaffinization.

5. The in-vitro method of any one of claims 1-4, wherein the sample has been scanned to acquire tissue images for co-registration, and/or image analysis purposes prior to step (i).

6. The in-vitro method of any one of claims 3-5, wherein the MALDI matrix is removed after step (i), preferably by washing with 70% ethanol.

7. The in-vitro method of any one of claims 1-6, wherein the sample is washed after step (ii).

8. The in-vitro method of any one of claims 1-7, wherein the in-vitro method further comprises the determination of the level of at least one additional biomarker, preferably the in-vitro method further comprises the determination of the level(s) of 1, 2, 3, 4, 5, 6, 7, 8, 9, 10 or more than 10 additional biomarkers in step (i).

9. The in-vitro method of any one of claims 1-8, wherein the disease is cancer and the first and/or the second biomarker is a tumor marker.

10. The in-vitro method of claim 9, wherein the first biomarker is adenosine monophosphate (AMP), wherein an increased AMP level is indicative for a diagnosis of cancer.

11. The in-vitro method of claim 9 or 10, wherein the second marker is HER2/neu (locus 17q11.2-q12) and the reference marker is the a satellite DNA sequence at the centromeric region of chromosome 17 (CEP17; locus 17p11.1-q11.1) and wherein a ratio of Her2/neu to CEP17 is indicative for a diagnosis of cancer.

12. The in-vitro method of any one of claims 1-11, wherein the first biomarker is adenosine monophosphate (AMP), the staining in step (ii) is hematoxylin and eosin stain (H&E stain), the second biomarker is HER2/neu and the reference marker is CEP17 and wherein an increase of the ratio of AMP to the ratio of HER2/neu and CEP17 is indicative for the presence of a cancer or a tumor precursor lesion.

13. The in-vitro method of any one of claims 1-12, wherein the disease is breast cancer, Barrett's cancer or gastroesophageal adenocarcinoma.

14. The in-vitro method of any one of claims 1-8, wherein the disease is a degenerative or inflammatory disease.
